(19) United States
(12) Patent Application Publication
(10) Pub. No.: US 2021/0249120 A1
(43) Pub. Date: Aug. 12, 2021

(54) SYSTEM AND METHOD FOR PROVIDING MODEL-BASED PREDICTIONS OF ACTIVELY MANAGED PATIENTS

(71) Applicant: KONINKLIJKE PHILIPS N.V., EINDHOVEN (NL)

(72) Inventors: Jennifer CAFFAREL, Eindhoven (NL); David LLOYD, Roswell, GA (US); Aleksandra TESANOVIC, Eindhoven (NL); Niels LAUTE, Venlo (NL)

(21) Appl. No.: 17/054,554

(22) PCT Filed: May 14, 2019
(86) PCT No.: PCT/EP2019/062308

§ 371 Date: Nov. 11, 2020

Related U.S. Application Data
(60) Provisional application No. 62/671,635, filed on May 15, 2018.

(51) Int. Cl. G16H 40/20; G16H 50/20; G16H 10/60; G06Q 40/08; G16H 20/00; G16H 50/70; G06Q 30/02; G06N 20/00

(52) U.S. Cl. CPC G16H 40/20; G16H 50/20; G16H 10/60; G06N 20/00; G16H 20/00; G16H 50/70; G06Q 30/0201; G06Q 40/08

(57) ABSTRACT

The present disclosure pertains to a system for providing model-based predictions of actively managed patients. In some embodiments, the system (i) obtains a collection of information related to a payer-attributed population of patients associated with a provider; (ii) extracts, from the collection of information, health insurance claims data, clinical data, process data, and patient encounter data; (iii) provides the health insurance claims data, clinical data, process data, and patient encounter data to a machine learning model to train the machine learning model; (iv) causes the machine learning model to predict familiarity values associated with patients of the population of patients; and (v) generates a provider assessment based on the familiarity values and the collection of information.

### BACKGROUND

### 1. Field

The present disclosure pertains to a system and method for providing model-based predictions related to patients associated with a provider, including predictions of patients actively managed by the provider or other patients associated with the provider.

### 2. Description of the Related Art

Healthcare networks working towards value-based care have to work with a range of healthcare providers to ensure that clinical, financial, and patient satisfaction goals are reached. This is often achieved by setting common performance indicators or quality measures common across the organization, and establishing performance assessments for the healthcare providers. Although automated and other computer-assisted provider performance assessment systems exist, such systems may assess the provider based on the payer-attributed population of patients associated with the provider and fail to distinguish the provider's performance with respect to a subset of the population actively managed by the provider, thus leading inherently to a misalignment of judgement and perception of performance. These and other drawbacks exist.

### SUMMARY

Accordingly, one or more aspects of the present disclosure relate to a system for providing model-based predictions of actively managed patients. The system comprises one or more processors configured by machine readable instructions and/or other components. The one or more processors are configured to: obtain, from one or more databases, a collection of information related to a payer-attributed population of patients associated with a provider; extract, from the collection of information, health insurance claims data, clinical data, process data, and patient encounter data; provide the health insurance claims data, clinical data, process data, and patient encounter data to a machine learning model to train the machine learning model; cause the machine learning model to predict familiarity values associated with patients of the population of patients; and generate a provider assessment based on the familiarity values and the collection of information.

Another aspect of the present disclosure relates to a method for providing model-based predictions of actively managed patients with a system. The system comprises one or more processors configured by machine readable instructions and/or other components. The method comprises: obtaining, with one or more processors, a collection of information related to a payer-attributed population of patients associated with a provider from one or more databases; extracting, with the one or more processors, health insurance claims data, clinical data, process data, and patient encounter data from the collection of information; providing, with the one or more processors, the health insurance claims data, clinical data, process data, and patient encounter data to a machine learning model to train the machine learning model; causing, with the one or more processors, the machine learning model to predict familiarity values associated with patients of the population of patients; and generating, with the one or more processors, a provider assessment based on the familiarity values and the collection of information.

Still another aspect of present disclosure relates to a system for providing model-based predictions of actively managed patients. The system comprises: means for obtaining a collection of information related to a payer-attributed population of patients associated with a provider from one or more databases; means for extracting health insurance claims data, clinical data, process data, and patient encounter data from the collection of information; means for providing the health insurance claims data, clinical data, process data, and patient encounter data to a machine learning model to train the machine learning model; means for causing the machine learning model to predict familiarity values associated with patients of the population of patients; and means for generating a provider assessment based on the familiarity values and the collection of information.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured for providing model-based predictions related to patients associated with a provider, in accordance with one or more embodiments.

FIG. 2 illustrates generation of provider assessments, in accordance with one or more embodiments.

FIG. 3 illustrates information communicated to providers based on model-based predictions, in accordance with one or more embodiments.

FIG. 4 illustrates a method for providing model-based predictions of actively managed patients, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

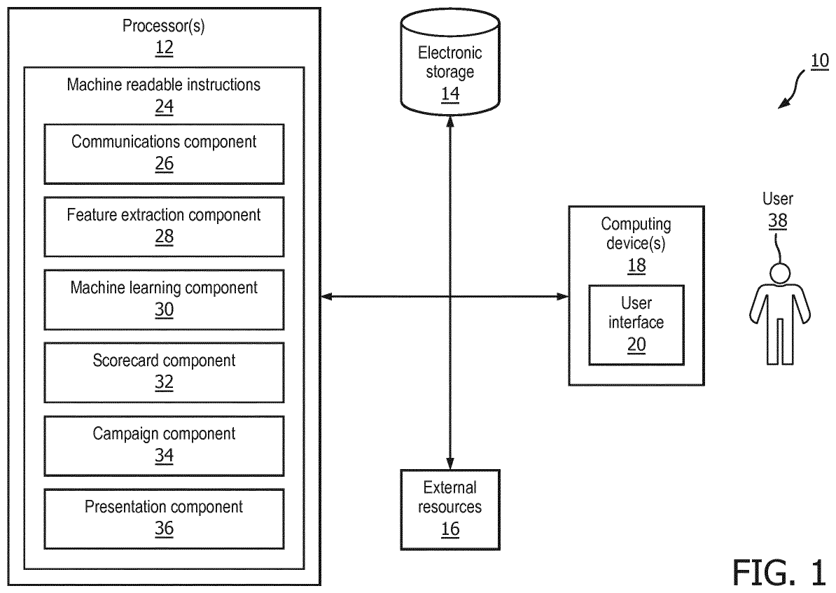

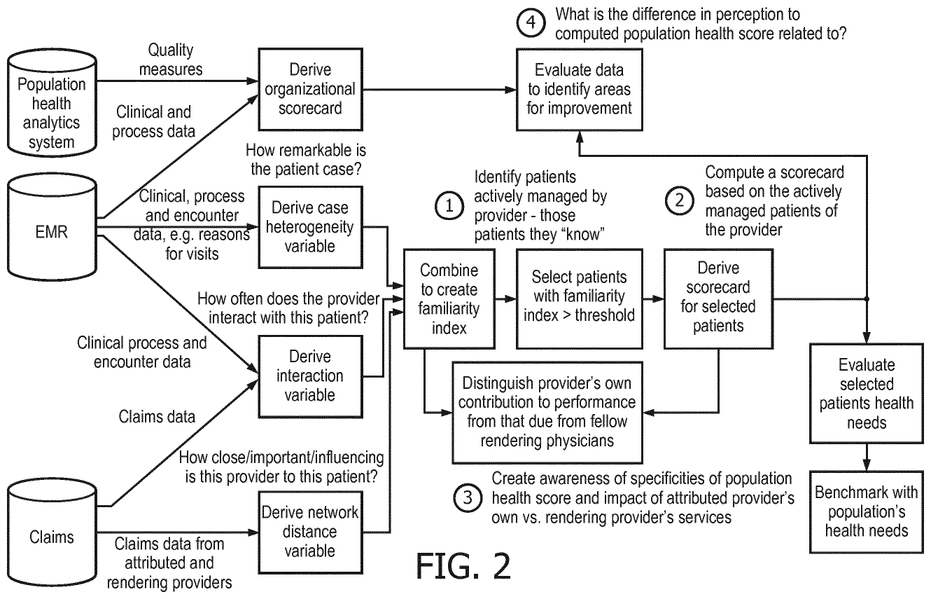

FIG. 1 is a schematic illustration of a system 10 configured for providing model-based predictions related to patients associated with a provider, in accordance with one or more embodiments. In some embodiments, system 10 is configured to identify patients actively managed by a provider (e.g., patients they “know”). In some embodiments, system 10 is configured to determine a first provider assessment based on data associated with actively managed patients of the provider (e.g., the subset of the population which the provider may perceive to be the population they manage). In some embodiments, system 10 is configured to determine a second provider assessment based on data associated with the entire payer-attributed population of patients associated with the provider (e.g. patients that the provider is responsible for managing according to the healthcare organization/payer). In some embodiments, the second provider assessment is indicative of outcome measurements (e.g., clinical, process, and financial outcome measurements) for all patients that have been attributed to the provider (e.g., even patients who are not actively being managed by the provider). In some embodiments, patients not actively managed may include patients who primarily seek care with other health care providers (e.g., other physicians, emergency departments, etc.), who rarely seek care, or who do not seek care at all. In some embodiments, system 10 is configured to create awareness of the specificities of a population health assessment and the impact of an attributed provider's own vs. a rendering provider's services. In some embodiments, system 10 is configured to determine one or more factors contributing to differences between the first provider assessment and the second provider assessment. In some embodiments, system 10 is configured to determine a feasibility of extending one or more proactive actions (e.g., learning actions) currently offered to the sub-population actively managed to the entire payer-attributed population. By way of a non-limiting example, FIG. 2 illustrates generation of provider assessments, in accordance with one or more embodiments. As shown in FIG. 2, system 10 determines the first provider assessment based on familiarity values associated with patients of the payer-attributed population of patients and the collection of information related to the payer-attributed population of patients associated with the provider.

Returning to FIG. 1, in some embodiments, system 10 is configured to generate one or more predictions related to familiarity values associated with patients of a population of patients, or perform other operations described herein via one or more prediction models. Such prediction models may include neural networks, other machine learning models, or other prediction models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

In some embodiments, system 10 comprises processors 12, electronic storage 14, external resources 16, computing device 18 (e.g., associated with user 38), or other components.

Electronic storage 14 comprises electronic storage media that electronically stores information (e.g., health insurance claims data, clinical data, process data, and patient encounter data). The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, etc.). In some embodiments, electronic storage 14 may be located in a server together with processors 12, in a server that is part of external resources 16, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processors 12, information received via processors 12 and/or graphical user interface 20 and/or other external computing systems, information received from external resources 16, and/or other information that enables system 10 to function as described herein.

External resources 16 include sources of information and/or other resources. For example, external resources 16 may include a population's electronic medical record (EMR), the population's electronic health record (EHR), or other information. In some embodiments, external resources 16 include health information related to the population. In some embodiments, the health information comprises demographic information, vital signs information, medical condition information indicating medical conditions experienced by individuals in the population, treatment information indicating treatments received by the individuals, care management information, and/or other health information. In some embodiments, external resources 16 include sources of information such as databases, websites, etc., external entities participating with system 10 (e.g., a medical records system of a health care provider that stores medical history information of patients, publicly and privately accessible social media websites), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 16 may be provided by resources included in system 10.

Processors 12, electronic storage 14, external resources 16, computing device 18, and/or other components of system 10 may be configured to communicate with one another, via wired and/or wireless connections, via a network (e.g., a local area network and/or the internet), via cellular technology, via Wi-Fi technology, and/or via other resources. It will be appreciated that this is not intended to be limiting, and that the scope of this disclosure includes embodiments in which these components may be operatively linked via some other communication media. In some embodiments, processors 12, electronic storage 14, external resources 16, computing device 18, and/or other components of system 10 may be configured to communicate with one another according to a client/server architecture, a peer-to-peer architecture, and/or other architectures.

Computing device 18 may be configured to provide an interface between user 38 and/or other users, and system 10. In some embodiments, computing device 18 is and/or is included in desktop computers, laptop computers, tablet computers, smartphones, smart wearable devices including augmented reality devices (e.g., Google Glass), wrist-worn devices (e.g., Apple Watch), and/or other computing devices associated with user 38, and/or other users. In some embodiments, computing device 18 facilitates presentation of a list of individuals assigned to a care manager, or other information. Accordingly, computing device 18 comprises a user interface 20. Examples of interface devices suitable for inclusion in user interface 20 include a touch screen, a keypad, touch sensitive or physical buttons, switches, a keyboard, knobs, levers, a camera, a display, speakers, a microphone, an indicator light, an audible alarm, a printer, tactile haptic feedback device, or other interface devices. The present disclosure also contemplates that computing device 18 includes a removable storage interface. In this example, information may be loaded into computing device 18 from removable storage (e.g., a smart card, a flash drive, a removable disk, etc.) that enables caregivers or other users to customize the implementation of computing device 18. Other exemplary input devices and techniques adapted for use with computing device 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device, devices that are part of external resources 16, electronic storage 14, or other devices.)

As shown in FIG. 1, processor 12 is configured via machine-readable instructions 24 to execute one or more computer program components. The computer program components may comprise one or more of a communications component 26, a feature extraction component 28, a machine learning component 30, a scorecard component 32, a campaign component 34, a presentation component 36, or other components. Processor 12 may be configured to execute components 26, 28, 30, 32, 34, or 36 by software; hardware; firmware; some combination of software, hardware, or firmware; or other mechanisms for configuring processing capabilities on processor 12.

It should be appreciated that although components 26, 28, 30, 32, 34, and 36 are illustrated in FIG. 1 as being co-located within a single processing unit, in embodiments in which processor 12 comprises multiple processing units, one or more of components 26, 28, 30, 32, 34, or 36 may be located remotely from the other components. The description of the functionality provided by the different components 26, 28, 30, 32, 34, or 36 described below is for illustrative purposes, and is not intended to be limiting, as any of components 26, 28, 30, 32, 34, or 36 may provide more or less functionality than is described. For example, one or more of components 26, 28, 30, 32, 34, or 36 may be eliminated, and some or all of its functionality may be provided by other components 26, 28, 30, 32, 34, or 36. As another example, processor 12 may be configured to execute one or more additional components that may perform some or all of the functionality attributed below to one of components 26, 28, 30, 32, 34, or 36.

In some embodiment, the present disclosure comprises means for obtaining, from one or more databases (e.g., electronic storage 14, external resources 16, etc.), a collection of information related to a payer-attributed population of patients associated with a provider. In some embodiments, such means for obtaining takes the form of communications component 26. In some embodiments, the collection of information includes all of the key administrative clinical data relevant to that patients care under a particular provider, such as demographics, progress notes, problems, medications, vital signs, past medical history, immunizations, laboratory data, radiology reports, or other information. In some embodiments, the collection of information includes digital equivalents of paper records, charts, or other patient records at a provider's office. In some embodiments, the collection of information includes treatment and medical history about one or more patients as collected by the individual provider, healthcare organization, or other entities. In some embodiments, the collection of information is related to all patients that have been attributed to the provider, even those who are not actively being managed by the provider. These may be patients who primarily seek care with other health care providers (e.g., other physicians, emergency departments, etc.), who rarely seek care, or who do not seek care at all.

In some embodiment, the present disclosure comprises means for extracting, from the collection of information, health insurance claims data, clinical data, process data, patient encounter data, or other information. In some embodiments, such means for extracting takes the form of feature extraction component 28. In some embodiments, health insurance claims data includes information gathered from medical bills or claims submitted by providers to government and private health insurers. In some embodiments, clinical data includes outcome measures reflective of the impact of the health care service or intervention on the health status of patients. For example clinical data may include the percentage of patients who died as a result of surgery (e.g., surgical mortality rates), the rate of surgical complications or hospital-acquired infections, or other information. In some embodiments, process data indicates what a provider does to maintain or improve health, either for healthy people or for those diagnosed with a health care condition. In some embodiments, process data includes specific steps in a process that lead (positively or negatively) to a particular outcome metric. For example, assuming the outcome measure is length of stay, a process metric for that outcome may be the amount of time that passes between when the provider ordered the discharge and when the patient was actually discharged. In some embodiments, patient encounter data may include information related to a patient's engagement with the healthcare system. For example, patient encounter data includes information related to (i) who provided the service, (ii) what service was provided, (iii) where the service was provided, (iv) when the service was provided, (v) why the service was provided, and (vi) other information.

In some embodiments, feature extraction component 28 is configured to determine, based on the health insurance claims data, clinical data, process data, patient encounter data, or other information, (i) an interaction parameter, (ii) a case heterogeneity parameter, (iii) a network distance parameter, or (iv) other parameters. In some embodiments, the interaction parameter is indicative of a frequency of interaction based on length of enrolment of a patient at a healthcare facility, a frequency of encounters during a predetermined amount of time (e.g., last year), consultations with multiple members of the same family, or other information. In some embodiments, more recent visits may be weighted more than earlier visits. In some embodiments, the case heterogeneity parameter is indicative of patient case heterogeneity. In some embodiments, the case heterogeneity parameter may influence the interaction parameter (e.g., balance) to reflect continuity and complexity of care (e.g., there is a different level of provider involvement when it comes to providing care for the same patient visiting 10 times for 10 different reasons, compared to the same patient visiting 10 times for the same reason). In some embodiments, feature extraction component 28 is configured to determine the case heterogeneity parameter based on one or more factors including reasons for encounters, co-morbidity profile, or other factors. In some embodiments, the network distance parameter is indicative of the positioning of a provider in a patient's greater care network. In some embodiments, the network distance parameter may indicate that patient may be subject to other providers' influences out of the provider's scope of control. In some embodiments, the network distance parameter may indicate the closer network to the physician in the provider group to account for services provided by “rendering physicians” on the account of the “attributed physician” in the health insurance claims data. In some embodiments, feature extraction component 28 is configured to determine which individual providers and/or services the patient has been in touch with over the predetermined amount of time.

In some embodiment, the present disclosure comprises means for providing the health insurance claims data, clinical data, process data, and patient encounter data (e.g., as obtained via feature extraction component 28) to a machine learning model to train the machine learning model. In some embodiments, such means for providing takes the form of machine learning component 30. In some embodiments, machine learning component 30 is configured to provide the interaction parameter, the case heterogeneity parameter, the network distance parameter, or other information to the machine learning model to train the machine learning model on the providers' dataset. In some embodiments, the machine learning model's training dataset is specific to the provider's population of patients.

In some embodiments, the machine learning model comprises a neural network (e.g., a feedforward neural network or other neural network). In some embodiments, the neural network comprises (i) one or more nodes of an input layer that correspond to the health insurance claims data, clinical data, process data, and patient encounter data, (ii) one or more nodes of an output layer that correspond to the familiarity values associated with patients of the population of patients, (iii) one or more nodes (or “neurons”) of at least one hidden layer, (iv) other components. In some embodiments, a feedforward neural network is configured such that information moves in only one direction, forward, from the input layer nodes, through the hidden layer nodes and to the output layer nodes. In some embodiments, the feedforward neural network may not include cycles or loops in the network. In some embodiments, machine learning component 30 is configured to determine a number of neurons (e.g., the predetermined number of neurons of a hidden layer or other neurons) in the neural network. In some embodiments, the neural network is configured to adjust weights associated with the neurons to minimize output error based on its assessment of feedback (e.g., user feedback, feedback self-generated by the neural network, etc.) or its assessment of its outputs (e.g., prior outputs against feedback or other outputs).

In some embodiments, machine learning component 30 comprises a multiple linear regression machine learning model. In some embodiments, the multiple linear regression machine learning model is configured to determine coefficients associated with inputs corresponding to the health insurance claims data, clinical data, process data, and patient encounter data based on at least a portion of the health insurance claims data, clinical data, process data, and patient encounter data. For example, 70% of the collection of information related to the payer-attributed population of patients associated with the provider may be used as a training data set and the remaining 30% of the collection of information may be used as testing samples.

For example, machine learning component 30 is configured to generate a linear regression model data based on at least a portion of the health insurance claims data, clinical data, process data, and patient encounter data as shown below:

Familiarity Value=β₀+β₁(interaction)+β₂ (case heterogeneity)+β₃(network distance)+εᵢ, wherein β₁, β₂, and β₃ represent coefficients associated with the interaction parameter, the case heterogeneity parameter, and the network distance parameter respectively.

In some embodiments, the present disclosure comprises means for causing the machine learning model to predict familiarity values associated with patients of the population of patients. In some embodiments, such means for causing takes the form of machine learning component 30. In some embodiments, the familiarity values are relative measures of familiarity within a providers' population (e.g., rather than a generic measure of familiarity across providers). In some embodiments, the familiarity values may facilitate identification of patients who the provider will have a lasting impression of (e.g., a regularly visiting patient with chronic conditions that the provider has been personally managing for years vs. a patient who only comes in for an episodic consultation for minor non-recurring conditions).

In some embodiments, the present disclosure comprises means for generating a provider assessment based on the familiarity values and the collection of information. In some embodiments, such means for generating takes the form of scorecard component 32. In some embodiments, the provider assessment is configured to provide (e.g., at a high level) an overview of long-term and strategic outcomes improvement goals for the population of patients associated with the provider (e.g., reduce readmissions, increase average patient satisfaction, and reduce average or turnaround times). In some embodiments, the provider assessment is configured to combine electronic medical records, financial/billing, patient satisfaction data, or other information to track strategic goals. In some embodiments, the provider assessment is configured to evaluate provider performance on an organizational level.

In some embodiments, scorecard component 32 is configured to select a subset of the payer-attributed population of patients associated with the provider based on the predicted familiarity values associated with each patient of the population of patients exceeding a predetermined threshold. In some embodiments, the subset may be indicative of patients actively managed by the provider. In some embodiments, scorecard component 32 is configured to generate a first provider assessment based on the collection of information corresponding to the subset of the payer-attributed population of patients associated with the provider (e.g., patients actively managed). In other words, scorecard component 32 is configured to generate the first provider assessment without the use of the collection of information corresponding to patients not included in the subset (e.g., patients not actively managed). In some embodiments, the first provider assessment is indicative of actual performance as perceived by the provider themselves.

In some embodiments, scorecard component 32 is configured to generate a second provider assessment (i) based on the collection of information and (ii) without using the predicted familiarity values. As such, the second provider scorecard is indicative of the provider's performance with respect to the entire payer-attributed population of patients associated with the provider.

By way of a non-limiting example, Table 1 illustrates provider assessments, in accordance with one or more embodiments. As shown in Table 1, quality measures are used to assess clinical, financial and process outcomes. In some embodiments, providers are benchmarked against organizational targets and their peers. In some embodiments, the provider assessment (e.g., the first provider assessment) distinguishes the providers' efforts in the part of the sub-population they actively manage.

TABLE 1

| | | |
|---|---|---|
| | Score on Total | Score on Actively |
| Quality Measure | Population | Managed Population |

Care Delivery
Total Cost of Care - PMPM
Avoidable Hospital
Admissions
Avoidable Hospital
Re-admissions
Preventive Care
Blood Pressure >1 yr or
Not Documented
Blood Pressure >140/90
(Last Value)
Tobacco Use: Screening
and Cessation Intervention
Female Age 45-54:
Mammogram >1 yrs or
Not Documented
Diabetes
Hemoglobin A1c Poor Control
Foot Exam
Eye Exam

In some embodiments, scorecard component 32 is configured to identify areas of focus needed to achieve optimal results in parts of the population not actively managed by the provider. In some embodiments, scorecard component 32 is configured to generate a personalized provider patient population needs assessment. In some embodiments, the personalized provider patient population needs assessment is indicative of the health and needs of the population beyond the organizational goals. In some embodiments, the personalized provider patient population needs assessment may support communication between the provider and organization on pragmatic strategic and operational decision making that could directly support an individual provider to meet their specific population's needs.

In some embodiments, campaign component 34 is configured to identify, based on a comparison of the first provider assessment and the second provider assessment, one or more patients (i) not actively managed by the provider and (ii) requiring the provider's attention. In some embodiments, campaign component 34 is configured to generate one or more care plans for the identified one or more patients.

In some embodiments, campaign component 34 is configured to obtain patient characteristics information associated with the subset of the population (actively managed patients). In some embodiments, the patient characteristics information include patients' clinical and demographic information. In some embodiments, patients' clinical and demographic information comprises one or more of an age, a gender, a primary diagnosis, a time since primary diagnosis, a number of secondary diagnosis, a frailty index, a 30-days readmissions risk score, one or more lab test results, a weight, a body mass index, or other information.

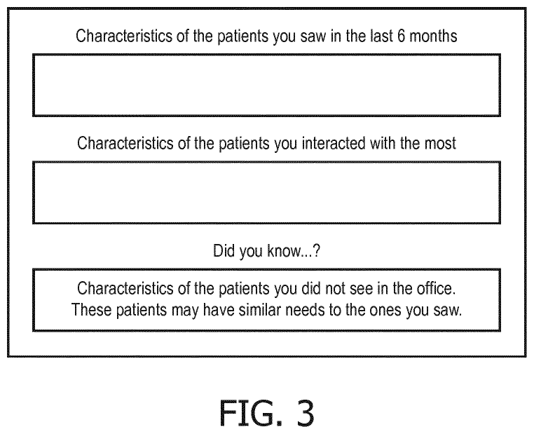

In some embodiments, campaign component 34 is configured to perform one or more queries (e.g., in a database associated with a healthcare organization, an accountable care organization, etc.) based on the patient characteristics information associated with the subset of the population to identify similar individuals (i) having similar patient characteristics information and (ii) not being currently managed by the provider. In some embodiments, campaign component 34 is configured to generate an outreach campaign to the similar individuals such that the similar individuals are managed by the provider. By way of a non-limiting example, FIG. 3 illustrates information communicated to providers based on model-based predictions, in accordance with one or more embodiments. As shown in FIG. 3, campaign component 34 is configured to identify patients having needs similar to patients currently managed by a provider. In FIG. 3, campaign component 34 provides patient characteristics information associated with individuals similar to those currently managed by the provider.

Returning to FIG. 1, in some embodiments, campaign component 34 is configured to determine an effect caused by one or more proactive actions on one or more (first) provider assessment constituents. In some embodiments, the proactive actions may currently be offered to the subset of the population. In some embodiments, the effect may include an improvement to one or more constituents of the (first) provider assessment. In some embodiments, campaign component 34 is configured to determine updated values corresponding to one or more constituents of the second provider assessment responsive to the proactive actions being extended to patients not currently included in the subset of the population (e.g., patients not actively managed). In some embodiments, campaign component 34 is configured to provide the updated values corresponding to one or more constituents of the second provider assessment to scorecard component 32 to determine an updated provider assessment. In some embodiments, campaign component 34 is configured to determine a difference between the second provider assessment and the updated provider assessment. In some embodiments, campaign component 34 is configured to determine a feasibility of extending the proactive actions to patients not currently included in the subset of the population (e.g., patients not actively managed) based on the determined difference.

In some embodiments, presentation component 36 is configured to effectuate, via user interface 20, the first provider assessment, the second provider assessment, familiarity values associated with patients of the population of patients, or other information. In some embodiments, presentation component 36 is configured to effectuate, via user interface 20, patient characteristics information associated with the similar individuals. In some embodiments, presentation component 36 is configured to effectuate, via user interface 20, the feasibility of extending the proactive actions to patients not currently included in the subset of the population (i.e., patients not actively managed).

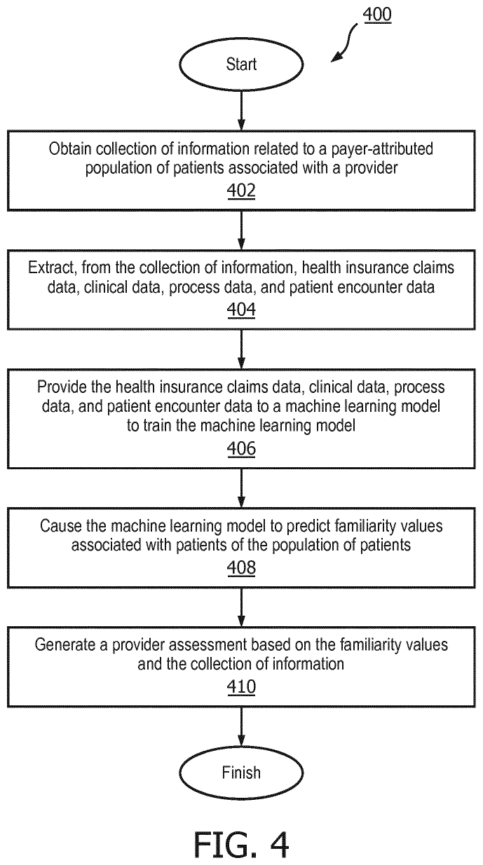

FIG. 4 illustrates a method 400 for providing model-based predictions of actively managed patients, in accordance with one or more embodiments. Method 400 may be performed with a system. The system comprises one or more processors, or other components. The processors are configured by machine readable instructions to execute computer program components. The computer program components include a communications component, a feature extraction component, a machine learning component, a scorecard component, a campaign component, a presentation component, or other components. The operations of method 400 presented below are intended to be illustrative. In some embodiments, method 400 may be accomplished with one or more additional operations not described, or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, or other mechanisms for electronically processing information). The devices may include one or more devices executing some or all of the operations of method 400 in response to instructions stored electronically on an electronic storage medium. The processing devices may include one or more devices configured through hardware, firmware, or software to be specifically designed for execution of one or more of the operations of method 400.

At an operation 402, a collection of information related to a payer-attributed population of patients associated with a provider is obtained from one or more databases. In some embodiments, operation 402 is performed by a processor component the same as or similar to communications component 26 (shown in FIG. 1 and described herein).

At an operation 404, health insurance claims data, clinical data, process data, and patient encounter data are extracted from the collection of information. In some embodiments, operation 404 is performed by a processor component the same as or similar to feature extraction component 28 (shown in FIG. 1 and described herein).

At an operation 406, the health insurance claims data, clinical data, process data, and patient encounter data are provided to a machine learning model to train the machine learning model. In some embodiments, operation 406 is performed by a processor component the same as or similar to machine learning component 30 (shown in FIG. 1 and described herein).

At an operation 408, the machine learning model is caused to predict familiarity values associated with patients of the population of patients. In some embodiments, operation 408 is performed by a processor component the same as or similar to machine learning component 30 (shown in FIG. 1 and described herein).

At an operation 410, a provider assessment is generated based on the familiarity values and the collection of information. In some embodiments, operation 410 is performed by a processor component the same as or similar to scorecard component 32 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A system for providing model-based predictions of actively managed patients, the system comprising:
one or more processors configured by machine-readable instructions to:
obtain, from one or more databases, a collection of information related to a payer-attributed population of patients associated with a provider;
extract, from the collection of information, health insurance claims data, clinical data, process data, and patient encounter data;
provide the health insurance claims data, clinical data, process data, and patient encounter data to a machine learning model to train the machine learning model;
cause the machine learning model to predict familiarity values associated with patients of the population of patients; and
generate a provider assessment based on the familiarity values and the collection of information.

2. The system of claim 1, wherein the one or more processors are configured to:
select a subset of the payer-attributed population of patients associated with the provider based on the predicted familiarity values associated with each patient of the population of patients exceeding a predetermined threshold; and
generate a first provider assessment based on the collection of information corresponding to the subset of the payer-attributed population of patients associated with the provider.

3. The system of claim 2, wherein the one or more processors are configured to generate a second provider assessment (i) based on the collection of information and (ii) without using the predicted familiarity values.

4. The system of claim 3, wherein the one or more processors are configured to
identify, based on a comparison of the first provider assessment and the second provider assessment, one or more patients (i) not actively managed by the provider and (ii) requiring the provider's attention;
generate one or more care plans for the identified one or more patients.

5. The system of claim 2, wherein the one or more processors are configured to:
obtain patient characteristics information associated with the subset of the payer-attributed population;
perform one or more queries based on the patient characteristics information associated with the subset of the payer-attributed population to identify similar individuals (i) having similar patient characteristics information and (ii) not being currently managed by the provider; and
generate an outreach campaign to the similar individuals to facilitate care management of the similar individuals by the provider.

6. A method for providing model-based predictions of actively managed patients, the method comprising:
obtaining, with one or more processors, a collection of information related to a payer-attributed population of patients associated with a provider from one or more databases;
extracting, with the one or more processors, health insurance claims data, clinical data, process data, and patient encounter data from the collection of information;
providing, with the one or more processors, the health insurance claims data, clinical data, process data, and patient encounter data to a machine learning model to train the machine learning model;
causing, with the one or more processors, the machine learning model to predict familiarity values associated with patients of the population of patients; and
generating, with the one or more processors, a provider assessment based on the familiarity values and the collection of information.

7. The method of claim 6, further comprising:
selecting, with the one or more processors, a subset of the payer-attributed population of patients associated with the provider based on the predicted familiarity values associated with each patient of the population of patients exceeding a predetermined threshold; and
generating, with the one or more processors, a first provider assessment based on the collection of information corresponding to the subset of the payer-attributed population of patients associated with the provider.

8. The method of claim 7, further comprising generating, with the one or more processors, a second provider assessment (i) based on the collection of information and (ii) without using the predicted familiarity values.

9. The method of claim 8, further comprising:
identifying, with the one or more processors, one or more patients (i) not actively managed by the provider and (ii) requiring the provider's attention based on a comparison of the first provider assessment and the second provider assessment; and
generating, with the one or more processors, one or more care plans for the identified one or more patients.

10. The method of claim 7, further comprising:
obtaining, with the one or more processors, patient characteristics information associated with the subset of the payer-attributed population;
performing, with the one or more processors, one or more queries based on the patient characteristics information associated with the subset of the payer-attributed population to identify similar individuals (i) having similar patient characteristics information and (ii) not being currently managed by the provider; and
generating, with the one or more processors, an outreach campaign to the similar individuals to facilitate care management of the similar individuals by the provider.

11. A system for providing model-based predictions of actively managed patients, the system comprising:
means for obtaining a collection of information related to a payer-attributed population of patients associated with a provider from one or more databases;
means for extracting health insurance claims data, clinical data, process data, and patient encounter data from the collection of information;
means for providing the health insurance claims data, clinical data, process data, and patient encounter data to a machine learning model to train the machine learning model;
means for causing the machine learning model to predict familiarity values associated with patients of the population of patients; and
means for generating a provider assessment based on the familiarity values and the collection of information.

12. The system of claim 11, further comprising:
means for selecting a subset of the payer-attributed population of patients associated with the provider based on the predicted familiarity values associated with each patient of the population of patients exceeding a predetermined threshold; and
means for generating a first provider assessment based on the collection of information corresponding to the subset of the payer-attributed population of patients associated with the provider.

13. The system of claim 12, further comprising means for generating a second provider assessment (i) based on the collection of information and (ii) without using the predicted familiarity values.

14. The system of claim 13, further comprising:
means for identifying one or more patients (i) not actively managed by the provider and (ii) requiring the provider's attention based on a comparison of the first provider assessment and the second provider assessment; and
means for generating one or more care plans for the identified one or more patients.

15. The system of claim 12, further comprising:
means for obtaining patient characteristics information associated with the subset of the payer-attributed population;
means for performing one or more queries based on the patient characteristics information associated with the subset of the payer-attributed population to identify similar individuals (i) having similar patient characteristics information and (ii) not being currently managed by the provider; and
means for generating an outreach campaign to the similar individuals to facilitate care management of the similar individuals by the provider.
